(19) United States
(12) Patent Application Publication
(10) Pub. No.: US 2016/0165935 A1
(43) Pub. Date: Jun. 16, 2016

(54) SYSTEM AND METHOD FOR FORMULATING A FOODSTUFF

(71) Applicant: COMMONWEALTH SCIENTIFIC AND INDUSTRIAL RESEARCH ORGANISATION, Campbell, Australian Capital Territory (AU)

(72) Inventors: Andrew EDDY, North Ryde (AU); Conor DELAHUNTY, North Ryde (AU); Ingrid APPELQVIST, Dural (AU); Leif LUNDIN, Werribee (AU); Graham Terence EYRES, Dunedin (NZ)

(21) Appl. No.: 14/905,589

(22) PCT Filed: Jul. 17, 2014
(86) PCT No.: PCT/AU2014/000732

§ 371 Date: Jan. 15, 2016

(30) Foreign Application Priority Data
Jul. 17, 2013 (AU) 2013902660

(51) Int. Cl. A23L 1/22; G01N 33/02; G06Q 50/00

(52) U.S. Cl. CPC A23L 1/22; G01N 33/02; A23V 2002/00

(57) ABSTRACT

A method of formulating a foodstuff includes determining a target flavour intensity of the foodstuff, determining one or more concentrations of one or more chemical stimuli to achieve the target flavour intensity of the foodstuff according to a flavour model, determining one or more concentrations of one or more ingredients of the foodstuff to achieve the determined concentrations of the one or more chemical stimuli, and formulating the foodstuff according to the determined concentrations of the one or more ingredients. The flavour model includes one or more stimulus intensity functions, each stimulus intensity function defining a relationship between a concentration of a chemical stimulus and a sensory stimulus intensity, and a flavour intensity function defining a relationship between a sensory stimulus intensity and a flavour intensity.

### FIELD OF THE INVENTION

The present invention relates to formulation of food. In particular, although not exclusively, the invention relates to formulating the flavour composition of a foodstuff based upon a flavour model.

### BACKGROUND TO THE INVENTION

Overconsumption of fat, sugar and salt is well known to contribute to a high incidence of obesity and chronic disease, and is a serious problem in the western world. Manufactured food products are, however, notorious for including high levels of fat, sugar and salt, and so there are currently efforts to reduce levels of these ingredients in manufactured food, and to reduce consumption of these ingredients generally.

However, a problem with simply cutting back on fat, sugar and/or salt in a food product is that a compromise in flavour and thus consumer liking is likely to occur. That in turn can lead to reduced sales of the food product.

In an attempt to alleviate this problem, reformulation of food products according to the prior art often includes reformulating a food product using expert taste panels and/or expert product development staff. In such case, reformulation is typically carried out by trial and error, where several different reformulations are compared until an acceptable formulation is found.

A problem with reformulating food using expert panels or product development staff is that this process is generally expensive and time consuming. In particular, as trial and error is used in the reformulation, or at best prior experience of a developer, it is likely that few of the reformulations provide a good balance between flavour and balance of ingredients. Additionally, taste panels are very labour intensive and thus costly.

A further problem with reformulating food using experts is that each product is reformulated and evaluated individually; and formulation of one product type is not easily transferred to formulation of other product types. Thus, investments of time and money increase as the number of food products increase.

Accordingly, there is a need for an improved system and method for formulating the flavour composition of a foodstuff.

It would be desirable to provide improvements and advantages over the above described prior art, and/or overcome or alleviate one or more of the above described disadvantages of the prior art, and/or provide a useful commercial choice.

### SUMMARY OF THE INVENTION

According to a first aspect, the invention provides a method of formulating the flavour composition of a foodstuff, the method including:

determining a target flavour intensity of the foodstuff;

determining one or more concentrations of one or more chemical stimuli to achieve the target flavour intensity of the foodstuff according to a flavour model, wherein the flavour model includes one or more stimulus intensity functions, each stimulus intensity function defining a relationship between a concentration of a chemical stimulus and a sensory stimulus intensity, and a flavour intensity function defining a relationship between the sensory stimulus intensities and a flavour intensity;

determining one or more concentrations of one or more ingredients of the foodstuff to achieve the determined concentrations of the one or more chemical stimuli; and

formulating the foodstuff according to the determined concentrations of the one or more ingredients.

Preferably one or more of the stimulus intensity functions and/or the flavour intensity function comprises a substantially s-shaped function. A sigmoid function such as a Gompertz function may be particularly preferable although various asymmetric or asymptotic functions may be suitable.

In some embodiments, the method further comprises e.g. reformulating a foodstuff, by:

predicting an original flavour intensity of an original foodstuff based at least in part on original concentrations of one or more ingredients of the original foodstuff and the flavour model;

wherein formulating the foodstuff according to the determined concentrations of the one or more ingredients comprises adjusting concentrations of the one or more ingredients in the original foodstuff.

The one or more ingredients may include a first ingredient and a second ingredient.

In some embodiments, the determined concentration of the first ingredient is higher than the original concentration of the first ingredient, and the reformulated concentration of the second ingredient is lower than the original concentration of the second ingredient.

Preferably, the target flavour intensity comprises a flavour intensity range.

The flavour intensity range may include the original flavour intensity.

In some embodiments, for a reformulated foodstuff a first ingredient of the one or more ingredients may have a negligible original concentration and a non-negligible reformulated concentration.

In some embodiments, the first ingredient is added to suppress a perceived stimulus intensity of another ingredient of the foodstuff.

In some embodiments, the one or more concentrations are determined further according to one or more desired foodstuff characteristics.

The one or more desired foodstuff characteristics may be selected from a group including but not limited to: reducing a concentration of an ingredient; increasing a concentration of an ingredient; and maintaining a concentration of an ingredient within a range.

Typically, the flavour model includes a taste component and an aroma component. Accordingly, it is preferable that the flavour model includes a plurality of stimulus intensity functions, at least one of which pertains to a taste stimulus and one of which pertains to an aromatic stimulus.

Typically, the flavour model is at least partly generated based upon empirical data.

Preferably, the flavour model is independent of a type of the foodstuff, where type refers generally to the food matrix format or structure (e.g. solid, liquid, semi-solid).

In some embodiments, the flavour model further includes factors that may impact flavour intensity. These factors may represent at least one of, for example, chemical heat, chemical cooling, temperature, lubricity, astringency, thickness, and creaminess.

In some embodiments, the flavour model further includes a factor representing a flavour release characteristic. Flavour release characteristics may be represented as e.g. ‘fast’ or ‘slow’ or may be determined according to a property of the foodstuff. For example, a particular flavour release characteristic may apply for a foodstuff coated in sugar, which, on consumption is quickly dissolved in saliva and the sweet taste perceived by the consumer. This would be different to the flavour release characteristic applicable to a foodstuff with the same quantity of sugar distributed homogenously throughout the foodstuff, where mastication is required to dissolve the sugar in saliva in order for the sweet taste to be perceived. These flavour release characteristics influence perceived flavour intensity.

The one or more ingredients may include one or more of: a fat; a sugar or sweetener; and a salt.

The one or more ingredients may include a pharmaceutical, a nutraceutical product or a nutritionally functional ingredient, and another ingredient for suppressing a flavour intensity associated with the pharmaceutical, the nutraceutical product or the functional ingredient. Functional ingredients can, for example, be present in so-called functional food, e.g. food fortified with vitamins and/or micro-nutrients.

According to a second aspect, the invention provides a system for formulating a foodstuff, the system comprising:

a data interface;

a processor coupled to the data interface; and

a formulation module comprising memory coupled to the processor, the formulation module including instruction code executable by the processor for:
determining one or more concentrations of one or more chemical stimuli to achieve a target flavour intensity of a foodstuff according to a flavour model, wherein the flavour model includes one or more stimulus intensity functions, each stimulus intensity function defining a relationship between a concentration of a chemical stimulus and a sensory stimulus intensity, and a flavour intensity function defining a relationship between a sensory stimulus intensity and a flavour intensity;
determining one or more concentrations of one or more ingredients of the foodstuff to achieve the determined concentrations of the one or more chemical stimuli; and
providing the determined concentrations of the one or more ingredients on the data interface.

### BRIEF DESCRIPTION OF THE DRAWINGS

To assist in understanding the invention and to enable a person skilled in the art to put the invention into practical effect, various embodiments of the invention are described below by way of example only with reference to the accompanying drawings, in which:

FIG. 1 diagrammatically illustrates a flavour model, according to an embodiment of the present invention;

FIG. 2 illustrates the sigmoid function relationship between sucrose concentration and perceived sweetness intensity, according to an embodiment of the present invention;

FIG. 3 illustrates a predicted flavour intensity response, according to an embodiment of the present invention;

FIG. 4 illustrates a method of reformulating a foodstuff, according to an embodiment of the present invention;

FIG. 5 illustrates a flavour model for reformulating a foodstuff, according to an embodiment of the present invention.

FIG. 6 diagrammatically illustrates a system, according to an embodiment of the present invention.

Those skilled in the art will appreciate that minor deviations from the layout of components as illustrated in the drawings will not detract from the proper functioning of the disclosed embodiments of the present invention.

### DETAILED DESCRIPTION OF THE INVENTION

Embodiments of the present invention comprise systems and methods for formulating or reformulating a foodstuff and more specifically, the flavour composition of a foodstuff. Elements of the invention are illustrated in concise outline form in the drawings, showing only those specific details that are necessary to the understanding of the embodiments of the present invention, but so as not to clutter the disclosure with excessive detail that will be known to those of ordinary skill in the art in light of the present description.

In this patent specification, adjectives such as first and second, left and right, front and back, top and bottom, etc., are used solely to define one element or method step from another element or method step without necessarily requiring a specific relative position or sequence that is described by the adjectives. Words such as “comprises” or “includes” are not used to define an exclusive set of elements or method steps. Rather, such words merely define a minimum set of elements or method steps included in a particular embodiment of the present invention.

The reference to any prior art in this specification is not, and should not be taken as, an acknowledgement or any form of suggestion that the prior art forms part of the common general knowledge.

According to one aspect, the invention resides in a method of formulating a foodstuff, the method including: determining a target flavour intensity of the foodstuff; determining one or more concentrations of one or more chemical stimuli to achieve the target flavour intensity of the foodstuff according to a flavour model, wherein the flavour model includes one or more stimulus intensity functions, each stimulus intensity function defining a relationship between a concentration of a chemical stimulus and a sensory stimulus intensity, and a flavour intensity function defining a relationship between a sensory stimulus intensity and a flavour intensity; determining one or more concentrations of one or more ingredients of the foodstuff to achieve the determined concentrations of the one or more chemical stimuli; and formulating the foodstuff according to the determined concentrations of the one or more ingredients.

The term “foodstuff” refers generally to any edible composition, including but not limited to food products, pharmaceutical products, and nutraceutical products, or any composition associated with a taste or aroma, whether the product is intended to be consumed or not.

Advantages of certain embodiments of the present invention may include an ability to efficiently develop healthy foods that maintain their palatability and/or reformulate foods for improved quality and/or nutritional content without or at least minimising adverse effect on palatability. This can in turn improve public health through reduced consumption of, for example, energy, fat, sugar and salt, or increase consumption of vegetables or nutrient rich food ingredients.

Certain embodiments of the present invention may increase patient compliance with medical treatments, as embodiments of the present invention can be used to provide an increased palatability of medicaments.

Certain embodiments of the present invention enable a reduction in development cost of a food product, while maintaining product quality. In particular, the time and cost associated with formulating or reformulating a food product to achieve a target flavour intensity can be reduced, as the model optimises the concentrations/amounts of certain ingredients so that the food product under development may require manufacturing and validation of a formulation only once, or only a small number of times.

Certain embodiments of the present invention enable a cost of a foodstuff to be reduced, without (or with only minimally) compromising sensory quality. Ingredients can, for example, be chosen such that costs of ingredients are balanced relative to their flavour impact.

Furthermore, according to certain embodiments, the present invention can be used to suppress, or otherwise alter palatability of an undesirable flavour or counteract the effect of a sensory stimulus. This can include, for example, suppressing bitterness in a vegetable, or suppressing a flavour of a compound in a pharmaceutical product which is unpleasant.

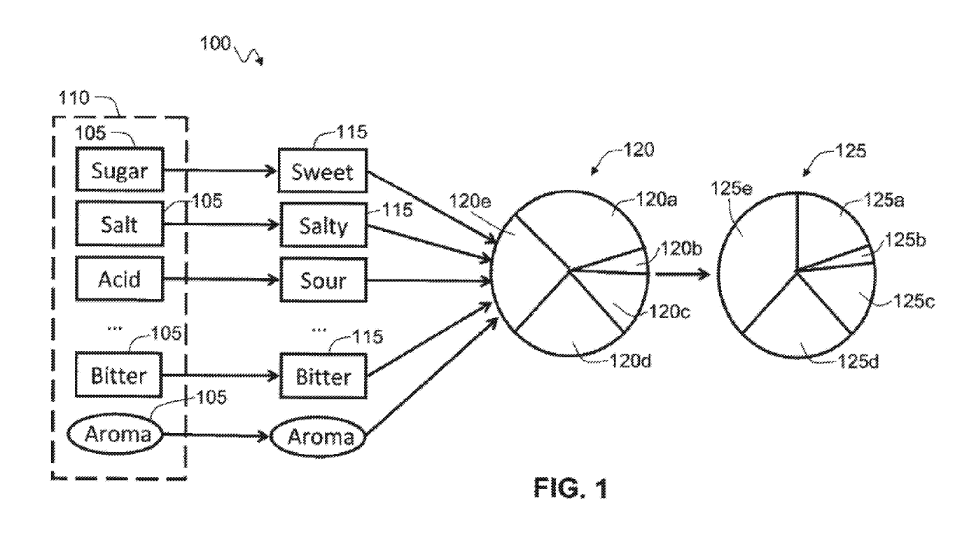

FIG. 1 diagrammatically illustrates a flavour model 100, according to an embodiment of the present invention. The flavour model 100 defines a relationship between concentrations of chemical stimuli, sensory stimulus intensity, flavour intensity and perceived mutual suppression of taste intensity, as discussed below.

The flavour model 100 includes a plurality of chemical stimulus concentration parameters 105, each chemical stimulus concentration parameter 105 of the plurality of chemical stimulus concentration parameters 105 relating to a concentration of a chemical stimulus. As an example, the chemical stimuli concentration parameters 105 include a parameter for sugar concentration, a parameter for salt concentration, a parameter for an acidic compound concentration, a parameter for a bitter compound concentration, and a parameter for an aroma.

As will be readily understood by the skilled addressee, not all of the chemical stimulus concentration parameters 105 illustrated in FIG. 1 need to be present in an alternative flavour model according to embodiments of the present invention. As an illustrative example, a flavour model used primarily for candy may not have or require a parameter for the bitter compound. Similarly, an alternative flavour model can include further chemical stimulus concentration parameters, such as a chemical stimulus concentration parameter for an aroma.

As shown in FIG. 1, the chemical stimuli that define the chemical stimulus concentration parameters 105 can comprise or be included in ingredients of a foodstuff 110. As will be understood by the skilled addressee, an ingredient of the foodstuff can comprise several sub-components, including one or more of the chemical stimuli, and other components. Similarly, a single chemical stimulus can be present in several ingredients of the foodstuff 110.

Each chemical stimulus parameter 105 is mapped to a taste intensity parameter 115 by a stimulus intensity sigmoid function. The stimulus intensity sigmoid functions dampen high and low intensity values, while leaving moderate intensity values relatively unchanged. Thus, a taste intensity parameter can reach saturation relatively quickly, wherein further increase in concentration of the corresponding chemical stimulus does not significantly increase taste intensity.

As will be readily understood by the skilled addressee, the term sigmoid curve is used to describe any substantially ‘S’ shaped function or variant thereof. Examples of sigmoid curves include Gompertz, Shifted Gompertz, Richards, Gumbell, and various Logistic and Laplacian functions. However, any suitable asymmetric, asymptotic function can be used, including a function based upon or solely defined by empirical data.

The taste intensity parameters 115 include e.g. a sweetness parameter, a saltiness parameter, a sourness parameter, a bitterness parameter and an aroma parameter, which are mapped to the parameter for sugar, the parameter for salt, the parameter for the acidic compound, the parameter for the bitter compound and the parameter for the aroma compound, respectively.

Examples of stimulus intensity sigmoid functions include:

I_{sweet}=e^{-9.45.e}^{^{−0.506.[sucrose]}} (Equation 1)

I_{salty}=e^{-4.22.e}^{^{−4.68.[NaCl]}} (Equation 2)

Iₐᵣₒₘₐ=e^{-2.66.e}^{^{−2.0.5.[aroma]}} (Equation 3)

where Equation 1 is for sweetness; Equation 2 is for saltiness; and Equation 3 is for aroma, and wherein [sucrose], [NaCl] and [aroma] are abbreviations for chemical stimulus concentration parameters 105, and I_{sweet}, I_{salty} and Iₐᵣₒₘₐ are perceived taste intensity parameters 115.

According to certain embodiments, the chemical stimulus parameters 105 are not mapped to taste intensity parameters 115 on a one-to-one basis. For example, a combined concentration of sucrose and glucose, or sucrose and an artificial sweetener, possibly weighted, can be mapped against sweetness. Similarly, a complex chemical stimulus parameter can be mapped to several taste intensity parameters 115.

The flavour model 100 further includes a flavour intensity parameter 120, the flavour intensity parameter 120 providing a composite flavour intensity of the foodstuff 110 which is determined according to both the taste and the aroma of the foodstuff.

The taste intensity parameters 115 are mapped to the flavour intensity parameter 120 by a weighting function, the weighting function defining an importance of each of the taste intensity parameters 115, and a flavour intensity sigmoid function. The flavour intensity sigmoid function is similar to the stimulus intensity sigmoid function above. Thus, the flavour intensity sigmoid function enables the flavour intensity parameter 120 to reach saturation without requiring each of the taste intensity parameters 115 to reach saturation. An example of a flavour intensity sigmoid function is:

I_{flavour}=e^{-4.1.e}^{^{−0.0.40.F}}^{sum} (Equation 4)

Fₛᵤₘ=0.22.I_{base}+0.31.I_{sweet}+0.24.I_{salty}+0.32.Iₐᵣₒₘₐ (Equation 5)

wherein I_{flavour} in Equation 4 is the flavour intensity parameter 120, and Fₛᵤₘ in Equation 5 is the weighting function, and I_{base} is a total taste intensity of sour and savoury components that are not modified.

The flavour intensity sigmoid function distorts very high and very low level weighted sums of the taste intensity parameters 115, while leaving almost undistorted the moderate level weighted sums of the taste intensity parameters 115. This uneven distortion has been identified as enabling better prediction of a flavour intensity of the foodstuff 110.

The flavour intensity parameter 120 includes first, second, third, fourth and fifth flavour intensity components 120a-120e, corresponding to weighted flavour intensities associated with the taste intensity parameters 115. The weighting of first, second, third, fourth and fifth flavour intensity components is generally performed using an estimate of a population-wide average. For example, levels of sweetness, sourness and aroma can be used to describe a fruity flavour. In this particular case, using data obtained from a panel of individuals who determined a ‘fruity’ flavour to be present in a flavour stimulus, a weighted sum of perceived flavour intensity can be calculated to be composed of 42% of a concentration for sweetness, 24% of a concentration for sourness, and 34% of a concentration for aroma.

The flavour model 100 further includes a context adjusted intensity parameter 125, the context adjusted intensity parameter 125 providing a context adjusted flavour intensity of the foodstuff 110. In particular, chemical stimuli can suppress and/or enhance each other. As an illustrative example, the presence of an aromatic chemical stimulus can enhance (or diminish) a perceived intensity of another chemical stimulus.

The flavour intensity parameter 120 is mapped to the context adjusted flavour intensity parameter 125 by a context mapping function. The context mapping function is advantageously a sigmoid function, similar to the flavour intensity sigmoid function as discussed above. Thus, the context mapping function enables a flavour intensity associated with a first chemical stimulus to be adjusted based upon a presence of a second chemical stimulus.

The context mapping function mimics a layer of distortion which results when flavour is deconstructed back into its component sensations. Such distortion can include e.g. mutual taste suppression and aroma-enhancement of taste.

The context adjusted intensity parameter 125 includes first, second, third, fourth, and fifth context adjusted intensity components 125a-125e, corresponding to the first, second, third, fourth and fifth flavour intensity components 120a-120e, adjusted according to context.

As can be seen in FIG. 1, the first context adjusted intensity component 125a has a smaller weight than the first flavour intensity component 120a. This can, for example, be due to a suppression of the first flavour intensity component 120a by the second, third, fourth and/or fifth flavour intensity component 120b to 120e.

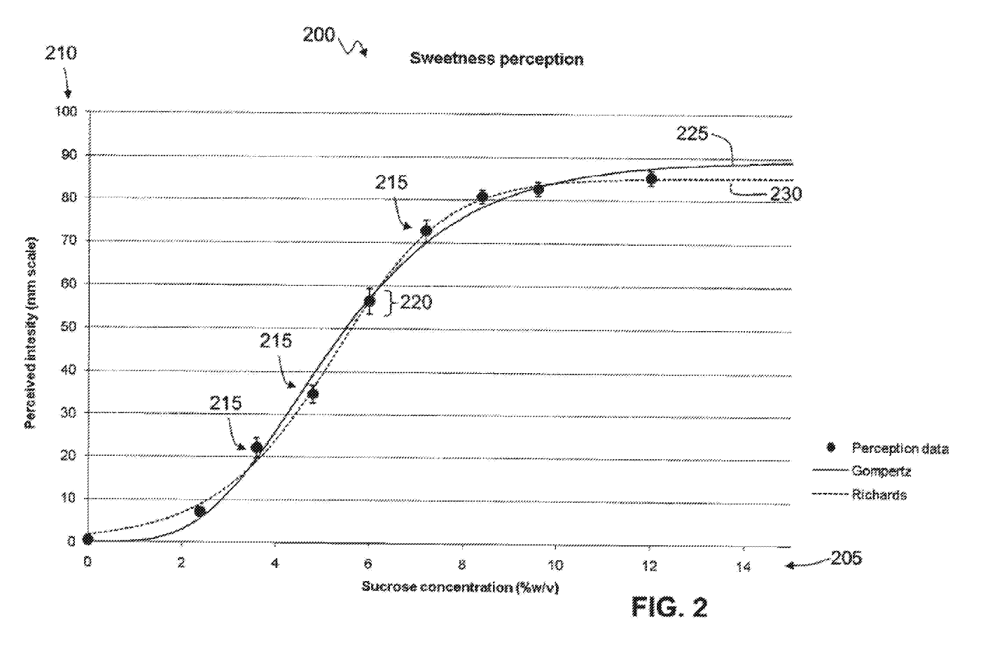

FIG. 2 illustrates a plot 200 of sucrose concentration 205 in a foodstuff against perceived sweetness intensity 210, according to an embodiment of the present invention. The plot 200 includes a plurality of data points 215, the data points 215 corresponding to perceived intensities for a plurality of sucrose samples. Each data point 215 corresponds to an average perceived sweetness intensity based upon mean data from a panel of human assessors. The data points 215 also include a standard error of a mean 220, illustrating a confidence of the respective data point 215.

The plot 200 further includes two alternative stimulus intensity sigmoid functions in the form of a Gompertz function 225 and a Richards function 230. Both the Gompertz function 225 and the Richards function 230 are fitted to the data points 215, and generally fit the data points 215 well. The Gompertz function 225 is, however, preferred over the Richards function 230, due to its mathematical simplicity, which simplifies modelling.

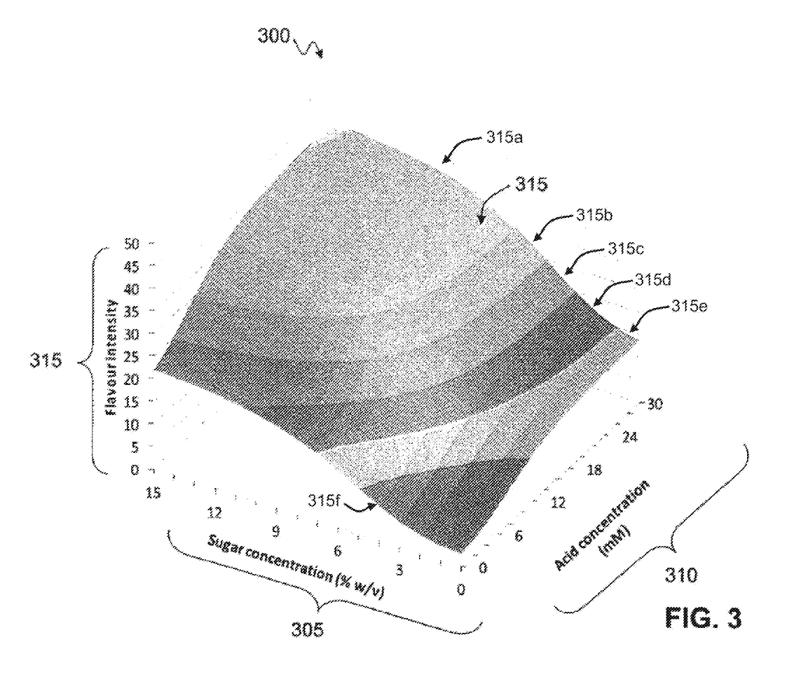

FIG. 3 illustrates a predicted overall flavour intensity response 300 of a foodstuff formulated using a flavour model, according to an embodiment of the present invention. The flavour model can be similar to the flavour model 100 of FIG. 1.

The flavour model maps a first concentration 305 of a first ingredient or chemical stimulus and a second concentration 310 of a second ingredient or chemical stimulus to a flavour intensity 315 of a foodstuff.

The flavour intensity 315 of the foodstuff generally corresponds to a perceived flavour intensity of the foodstuff by a typical person. However, as will be understood by a skilled addressee, the flavour model can be built for or adapted to suit a particular consumer or a segment or group of consumers, such as e.g. an ethnic group or age group, or a group that particularly enjoys or particularly dislikes specific types of foods.

The first and second concentrations 305, 310 are similar to the chemical stimulus concentration parameter 105, and the flavour intensity 315 is similar to the flavour intensity parameter 120. The flavour model incorporates first and second stimulus intensity sigmoid functions, associated with the first and second ingredients or chemical stimuli, and a flavour intensity sigmoid function defining a relationship between sensory stimulus intensities and a flavour intensity.

The flavour model further includes a plurality of flavour intensity bands 315a-f, each flavour intensity band 315a-f corresponding to a flavour intensity interval. According to certain embodiments, the flavour intensity bands 315a-f can correspond to subjective ranges, such as low flavour intensity, acceptable flavour intensity and good flavour intensity. According to alternative embodiments, the flavour intensity bands 315a-f can be evenly distributed over a flavour intensity.

The flavour model can be used to predict a flavour intensity of a foodstuff based upon concentrations of ingredients, or to predict a required concentration of one or more ingredients to achieve a target flavour intensity. Accordingly, the flavour model can be used to predict changes in flavour intensity of a foodstuff as concentrations of ingredients are changed, which can in turn be used to reformulate the foodstuff.

Reformulation of a foodstuff may be useful when it is desirable to reduce a concentration of one or more ingredients in the foodstuff, such as sugar, salt or fat, while maintaining a similar flavour intensity. Similarly, reformulation of a foodstuff can be used to increase a concentration of one or more ingredients in the foodstuff, such as vitamin rich or nutritionally functional ingredients, while maintaining a similar or desired flavour intensity.

According to certain embodiments, the flavour model can be applied generally across all types of food products. However, as will be readily understood by the skilled addressee, a flavour model can be optimised to suit a particular category of food, such as savoury or sweet foods, or a particular type of food, such as a tomato based pasta sauce.

The flavour model is typically derived from a detailed understanding of the human mechanisms of taste and/or aroma perception and the fusion of flavour, and parameters of the flavour model can be advantageously derived empirically.

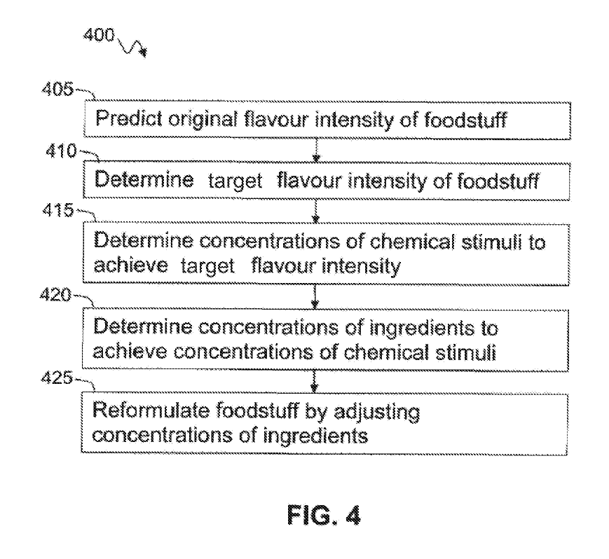

FIG. 4 illustrates a method 400 of reformulating a foodstuff, according to an embodiment of the present invention.

At step 405, an original flavour intensity of the foodstuff is either measured by a sensory panel, or predicted based at least in part on original concentrations of one or more ingredients and a flavour model. The flavour model can, for example, comprise a flavour model similar to the flavour models 100 described above with reference to FIG. 1. Thus, the flavour model may include a plurality of stimulus intensity sigmoid functions, each stimulus intensity sigmoid function defining a relationship between a concentration of a chemical stimulus and a sensory stimulus intensity, and a flavour intensity sigmoid function defining a relationship between sensory stimulus intensities and a flavour intensity.

As will be readily understood by the skilled addressee, the original flavour intensity of the foodstuff can alternatively be measured or already known. In such case, no prediction of the original flavour intensity is required.

At step 410, a target flavour intensity of the foodstuff is determined. As an illustrative example, determining a target flavour intensity of the foodstuff can comprise determining that the target flavour intensity of the foodstuff should not differ significantly from the original flavour intensity. This is particularly relevant when attempting to remove unnecessary amounts of salt, sugar and/or fat in a food, without significantly altering taste. As a further illustrative example, determining a target flavour intensity of the foodstuff comprises determining that the target flavour intensity of the foodstuff should be increased to an acceptable level.

At step 415, one or more reformulated concentrations of one or more chemical stimuli is determined to achieve the target flavour intensity of the foodstuff according to the flavour model.

According to certain embodiments, the reformulated concentrations are further determined according to one or more desired foodstuff characteristics. Examples of a desired foodstuff characteristic include e.g. a low level of salt, sugar and/or fat. In such case, the foodstuff can be reformulated to contain ingredients that meet the target flavour intensity while reducing salt, sugar and/or fat content in the foodstuff.

At step 420, one or more reformulated concentrations of one or more ingredients of the foodstuff is determined to achieve the determined reformulated concentrations of the one or more chemical stimuli.

At step 425, the foodstuff is reformulated by adjusting concentrations of the one or more ingredients in the original foodstuff to the reformulated concentrations.

As will be readily understood by the skilled addressee, the method 400 can be modified for generation of a new foodstuff rather than reformulation of an existing foodstuff. In such case, step 405 is omitted, and the new foodstuff is formulated using the “reformulated” concentrations.

According to certain embodiments, one or more steps of the method 400 can be repeated to predict an impact of an aroma on flavour intensity. However, according to other embodiments, aroma based ingredients can be considered together with flavour based ingredients.

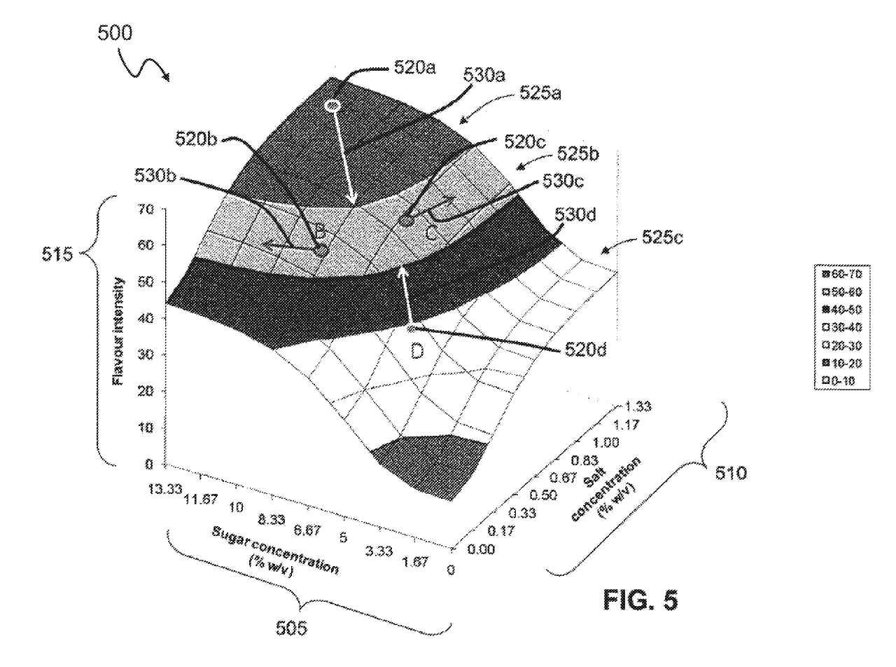

FIG. 5 illustrates a flavour model 500 for reformulating a foodstuff, according to an embodiment of the present invention.

The flavour model 500 is similar to the flavour model of FIG. 3, and maps a concentration of sugar 505 and a concentration of salt 510 to an overall flavour intensity 515 of the foodstuff.

An original flavour intensity of a first foodstuff is either measured by a sensory panel, or predicted based on original concentrations of the sugar and salt and the flavour model, as illustrated by first original flavour intensity point 520a. The first original flavour intensity point 520a lies in a saturated flavour intensity band 525a, which indicates that sugar and/or salt concentrations are unnecessarily high. As discussed above, the original flavour intensity of the first foodstuff can be measured or already known.

A target flavour intensity of the first foodstuff is then determined, as discussed above. In such case, a target flavour intensity in a lower end of the saturated flavour intensity band 525a can be achieved with a substantial reduction in salt and sugar, as illustrated by arrow 530a. Such a reformulation can provide a reduction of salt and sugar of approximately 30%, with minimal change to flavour intensity.

This is particularly relevant when attempting to remove excessive amounts of salt and sugar from a food, without significantly altering its flavour.

An original flavour intensity of a second foodstuff is measured or predicted, as discussed above, as illustrated by second original flavour intensity point 520b. The second original flavour intensity point 520b lies in a good flavour intensity band 525b. A target flavour intensity in the good flavour intensity band 525b can be achieved with a decreased salt concentration and an increase in sugar concentration, as illustrated by arrow 530b. Such a reformulation can provide a reduction of salt, while retaining flavour intensity.

An original flavour intensity of a third foodstuff is measured or predicted, as discussed above, as illustrated by third original flavour intensity point 520c. The third original flavour intensity point 520c also lies in the good flavour intensity band 525b. A target flavour intensity in the good flavour intensity band 525b can be achieved with a decreased sugar concentration and an increased salt concentration, as illustrated by arrow 530c. Such a reformulation can provide a reduction of sugar, while retaining flavour intensity.

Finally, an original flavour intensity of a fourth foodstuff is measured or predicted, as discussed above, as illustrated by fourth original flavour intensity point 520d. The fourth original flavour intensity point 520d lies in an unacceptable flavour intensity band 525c. A target flavour intensity in a lower end of the good flavour intensity band 525b can be achieved with an increase in sugar and salt concentrations, as illustrated by arrow 530d. Such a reformulation can provide an improvement in flavour intensity, without including unnecessary amounts of salt and sugar in the fourth foodstuff.

As illustrated above, once a target flavour intensity is determined, several possible combinations of concentrations of ingredients can be used to achieve the target flavour intensity. In such case, a further desired parameter can be identified for the foodstuff, such as including minimising a concentration of an ingredient, as discussed above, or minimising a cost of a combination of ingredients. In one embodiment, the predicted flavour intensity response of FIG. 3 may be modified, replacing ‘flavour intensity’ on the y-axis with cost. This enables the foodstuff to be optimised for flavour while satisfying an acceptable cost band. Representing the model in this way guides how far the composition of the foodstuff can be modified e.g. to minimise cost, before the flavour perception becomes noticeably different.

In some embodiments, cost relates to a monetary value. However other cost factors such as manufacturability, sustainability, water and carbon usage and energy consumption may be “cost” factors.

The foodstuff can then be reformulated by adjusting concentrations of the one or more ingredients in the original foodstuff to the reformulated concentrations. This may also be done using an iterative computational process in which cost constraints are applied to the flavour model.

According to certain embodiments, additional food characteristics that may or may not impact on flavour intensity can be included in the flavour models 100, 500. In particular, chemical heat (e.g. heat sensation from chilli, pepper, or mustard), chemical cooling (e.g cooling sensation from menthol), food temperature, texture (e.g. lubricity, astringency, thickness, or creaminess) and flavour release characteristics (e.g. taste or aroma release characteristics from a solid food matrix or from a surface of a food) can be incorporated into the flavour model.

For example, a foodstuff may be formulated to achieve a target flavour intensity while maximising creaminess or optimising astringency. Similarly, a food temperature can influence flavour intensity of a foodstuff, and can be considered when formulating the foodstuff.

According to certain embodiments, the present invention can be used on a variety of foodstuffs and for different purposes. In particular, certain embodiments of the present invention can be used to:

reformulate e.g. a tomato-based pasta sauce to maintain flavour while reducing salt and sugar content;

reformulate fruit juice blends to increase flavour, with reduced acid content (for dental health) and/or sugar content (for dental and metabolic health);

reformulate soups to maintain flavour while reducing salt content;

reformulate foods in a solid matrix, accounting for effect of texture;

formulate sausages to maintain flavour and decrease salt by targeted addition of trace savoury, sour, chemical heat and ‘fatty’ tastes;

formulate shortbread biscuits to maintain flavour and decrease salt by targeted addition of aroma and changes to tastant release rates;

formulate new products, based on predictive modelling of flavour intensity for ingredients and/or chemical stimulants having complex taste combinations;

predict an ingredient composition to achieve a target flavour intensity for a new product;

suppress or mask an undesired flavour, for example in a pharmaceutical or nutraceutical or food fortified with a functional ingredient;

formulate a pill, powder, paste or syrup, from a base of aroma, sweetness, saltiness with the intention of masking bitterness;

formulate flavour components of a ‘fortified’ beverage or food to suppress or mask bitter or otherwise unpleasant micro-nutrients or nutritionally functional ingredients, such as polyphenols; and

reformulate a food to target a preference of a specific group of consumers, such as a geographic, ethnic or cultural group.

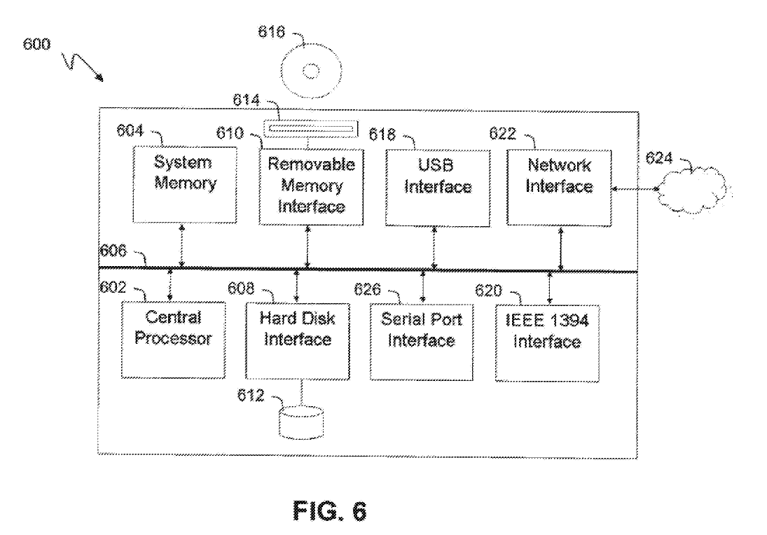

FIG. 6 diagrammatically illustrates a system 600, according to an embodiment of the present invention. The methods 400 and 500 of FIGS. 4 and 5 can be implemented using the system 600.

The system 600 includes a processor 602, a system memory 604 and a system bus 606 that couples various system components, including coupling the system memory 604 to the central processor 602. The system bus 606 may be any of several types of bus structures including a memory bus or memory controller, a peripheral bus, and a local bus using any of a variety of bus architectures. The structure of system memory 604 is well known to those skilled in the art and may include a basic input/output system (BIOS) stored in a read only memory (ROM) and one or more program modules such as operating systems, application programs and program data stored in random access memory (RAM).

The system 600 can also include a variety of interface units and drives for reading and writing data. The data can include, for example, concentrations of ingredients, parameters associated with flavour models, and any other associated data.

In particular, the system 600 may include a hard disk interface or solid state drive (SSD) interface 608 and a removable memory interface 610, respectively coupling a hard disk drive/SSD 612 and a removable memory drive 614 to the system bus 606. Examples of removable memory drives 614 include magnetic disk drives and optical disk drives. The drives and their associated computer-readable media, such as a Digital Versatile Disc (DVD) 616 provide non-volatile storage of computer readable instructions, data structures, program modules and other data for the computer system 600. A single hard disk drive/SSD 612 and a single removable memory drive 614 are shown for illustrative purposes only and with the understanding that the system 600 can include several similar drives. Furthermore, the system 600 can include drives for interfacing with other types of computer readable media.

The system 600 may include additional interfaces for connecting devices to the system bus 606. FIG. 6 shows a universal serial bus (USB) interface 618 which may be used to couple a device to the system bus 606. For example, an IEEE 1394 interface 620 may be used to couple additional devices to the system 600. Examples of additional devices include cameras for receiving images or video, or microphones for recording audio.

The system 600 can operate in a networked environment using logical connections to one or more remote computers or other devices, such as a server, a router, a network personal computer, a peer device or other common network node, a wireless telephone or wireless personal digital assistant. The system 600 includes a network interface 622 that couples the system bus 606 to a local area network (LAN) 624. Networking environments are commonplace in offices, enterprise-wide computer networks and home computer systems.

A wide area network (WAN), such as the Internet, can also be accessed by the system, for example via a modem unit connected to a serial port interface 626 or via the LAN 624.

Transmission and reception of data, such as ingredient concentration data, can be performed using the LAN 624, the WAN, or a combination thereof.

It will be appreciated that the network connections shown and described are exemplary and other ways of establishing a communications link between computers can be used.

The operation of the system 600 can be controlled by a variety of different program modules. Examples of program modules are routines, programs, objects, components, and data structures that perform particular tasks or implement particular abstract data types. The present invention may also be practiced with other computer system configurations, including hand-held devices, multiprocessor systems, microprocessor-based or programmable consumer electronics, network PCs, minicomputers, mainframe computers, personal digital assistants and the like. Furthermore, the invention may also be practiced in distributed computing environments where tasks are performed by remote processing devices that are linked through a communications network. In a distributed computing environment, program modules may be located in both local and remote memory storage devices.

In summary, advantages of certain embodiments of the present invention include an ability to efficiently develop healthy foods that maintain their palatability. This can in turn improve public health through reduced consumption of, for example, energy, fat, sugar and salt, or increase consumption of vegetables or nutrients. Alternatively/ additionally, the present invention may facilitate development of foods that have increased fibre and protein content, without altering significantly the palatability of the foodstuff.

Further, certain embodiments of the present invention enable a reduction in development cost of a food product, while maintaining product quality. In particular, a time and cost of the reformulation process to achieve a target formulation can be reduced, as certain embodiments only require manufacturing and validation of a formulation once, or a small number of times.

Certain embodiments of the present invention enable a cost of a foodstuff to be reduced, without (or minimally) compromising taste. Ingredients can, for example, be chosen such that costs of ingredients are balanced relative to their flavour impact.

Furthermore, according to certain embodiments, the present invention can be used to suppress, or otherwise alter palatability of an undesirable flavour. This can include, suppressing bitterness in a vegetable, or masking a flavour of a compound in a pharmaceutical product.

The above description of various embodiments of the present invention is provided for purposes of description to one of ordinary skill in the related art. It is not intended to be exhaustive or to limit the invention to a single disclosed embodiment. As mentioned above, numerous alternatives and variations to the present invention will be apparent to those skilled in the art of the above teaching. Accordingly, while some alternative embodiments have been discussed specifically, other embodiments will be apparent or relatively easily developed by those of ordinary skill in the art. Accordingly, this patent specification is intended to embrace all alternatives, modifications and variations of the present invention that have been discussed herein, and other embodiments that fall within the spirit and scope of the above described invention.

1. A method of formulating a foodstuff, the method including:
determining a target flavour intensity of the foodstuff;
determining one or more concentrations of, one or more chemical stimuli to achieve the target flavour intensity of the foodstuff according to a flavour model, wherein the flavour model includes:
one or more stimulus intensity sigmoid functions, each stimulus intensity sigmoid function defining a relationship between a concentration of a chemical stimulus and a sensory stimulus intensity, and
a flavour intensity sigmoid function defining a relationship between a sensory stimulus intensity and a flavour intensity;
determining one or more concentrations of one or more ingredients of the foodstuff to achieve the determined concentrations of the one or more chemical stimuli; and
formulating the foodstuff according to the determined concentrations of the one or more ingredients.

2. The method of claim 1, wherein one or more of the stimulus intensity sigmoid functions and/or the flavour intensity sigmoid function is selected from the group including an “S”-shaped, Gompertz, Shifted Gompertz, Richards, Gumbell, Logistic, Laplacian, Gamma, incomplete Gamma, Erlang and modified Erlang function.

3. The method of claim 1, further comprising:
predicting an original flavour intensity of an original foodstuff based at least in part on original concentrations of one or more ingredients of the original foodstuff and the flavour model; and
reformulating the original foodstuff according to the determined concentrations of the one or more ingredients by adjusting concentrations of the one or more ingredients in the original foodstuff.

4. The method of claim 3, wherein the one or more ingredients include a first ingredient and a second ingredient.

5. The method of claim 4, wherein the determined concentration of the first ingredient is higher than the original concentration of the first ingredient, and the determined concentration of the second ingredient is lower than the original concentration of the second ingredient.

6. The method according to claim 1, wherein the target flavour intensity comprises a flavour intensity range.

7. The method of claim 6, wherein the target flavour intensity range includes an original flavour intensity.

8. The method according to claim 3, wherein a first ingredient of the one or more ingredients has a negligible original concentration and a non-negligible reformulated concentration.

9. The method of claim 8, including increasing the first ingredient concentration to suppress a perceived stimulus intensity of another ingredient of the foodstuff.

10. (canceled)

11. The method according to claim 1, wherein the one or more concentrations are determined further according to one or more desired foodstuff characteristics selected from the group comprising: reducing a concentration of an ingredient; increasing a concentration of an ingredient; maintaining a concentration of an ingredient within a range; reducing a cost of the foodstuff.

12. The method according to claim 1, wherein the flavour model includes a taste component and an aroma component.

13. The method according to claim 1, wherein the flavour model is at least partly generated based upon empirical data.

14. The method according to claim 1, wherein the flavour model is independent of a type of the foodstuff.

15. The method according to claim 1, wherein the flavour model further includes factors representing at least one of: chemical heat, chemical cooling, temperature, lubricity, astringency, thickness, and creaminess.

16. The method according to claim 1, wherein the flavour model further includes a factor representing a flavour release characteristic.

17. The method according to claim 1, wherein the one or more ingredients include one of: a fat; a sugar or sweetener; and a salt.

18. The method according to claim 1, wherein the one or more ingredients include a pharmaceutical, a nutraceutical product or a functional ingredient, and another ingredient for suppressing a flavour intensity associated with the pharmaceutical, the nutraceutical product or the functional ingredient.

19. The method according to claim 1, wherein the flavour model includes a plurality of flavour intensity bands corresponding to a plurality of flavour intensity intervals for guiding reformulation of the foodstuff.

20. A system for formulating a foodstuff, the system comprising:
a data interface;
a processor coupled to the data interface; and
a formulation module comprising memory coupled to the processor, the formulation module including instruction code executable by the processor for:
determining one or more concentrations of one or more chemical stimuli to achieve a target flavour intensity of the foodstuff according to a flavour model, wherein the flavour model includes one or more stimulus intensity sigmoid functions, each stimulus intensity sigmoid function defining a relationship between a concentration of a chemical stimulus and a sensory stimulus intensity, and a flavour intensity sigmoid function defining a relationship between a sensory stimulus intensity and a flavour intensity;
determining one or more concentrations of one or more ingredients of the foodstuff to achieve the determined concentrations of the one or more chemical stimuli; and
providing the determined concentrations of the one or more ingredients on the data interface.

21. The system for formulating a foodstuff according to claim 20, wherein one or more of the stimulus intensity sigmoid functions and/or the flavour intensity sigmoid function is selected from the group including an “S”-shaped, Gompertz, Shifted Gompertz, Richards, Gumbell, Logistic, Laplacian, Gamma, incomplete Gamma, Erlang and modified Erlang function.
